(19) United States
(12) Patent Application Publication
(10) Pub. No.: US 2026/0022426 A1
(43) Pub. Date: Jan. 22, 2026

(54) METHOD FOR MONITORING AND EVALUATION OF BOWEL HEALTH IN PREMATURE NEWBORNS

(71) Applicant: Nitin Shashikant Chouthai, Novi, MI (US)

(72) Inventor: Nitin Shashikant Chouthai, Novi, MI (US)

(21) Appl. No.: 19/271,797

(22) Filed: Jul. 17, 2025

Related U.S. Application Data
(60) Provisional application No. 63/673,172, filed on Jul. 19, 2024.

(51) Int. Cl. C12Q 1/689; A61B 10/00; C12Q 1/6806; G01N 33/569

(52) U.S. Cl. CPC C12Q 1/689; A61B 10/0038; G01N 33/56972; A61B 2503/045; C12Q 1/6806; C12Q 2600/118; C12Q 2600/158

(57) ABSTRACT

Disclosed is a method for monitoring and evaluation of bowel health in premature newborns. The method involves collecting stool/fecal samples from premature newborns/babies and processing the sample using microbiomics, automated cell counting, flowcytometry, and RT PCR analysis using specific gene signature or RNA transcriptomics analysis to determine risk of necrotizing enterocolitis. The method allows evaluation and tracking of gut health without needing to draw a blood test and is a non-invasive method. The method diagnoses and prevents bowel inflammation, infection and necrotizing enterocolitis (NEC) that facilitates initiation of prompt therapy to limit morbidity and mortality in premature babies. The method facilitates early management of signs of NEC thereby helping to save lives of premature babies and infants having low birth weight.

### TECHNICAL FIELD

The present disclosure relates to identification of one or more disorders or conditions in infants and more particularly, the present invention relates to a method for monitoring and evaluation of bowel health in premature newborns.

### BACKGROUND

Necrotizing enterocolitis (NEC) is serious gastrointestinal disease in which the tissue lining the intestine becomes inflamed and dies. Babies born prematurely and infants having low birth weight are at high risk of NEC. It is a leading cause of morbidity and mortality in neonatal intensive care units. As several other clinical conditions mimic NEC, its diagnosis can be difficult and treatment is challenging. Currently, diagnosis is mainly based on physical examination, clinical symptoms, laboratory findings and imaging findings. The laboratory findings are based on blood tests and fecal tests whereas imaging findings are based on an abdominal X-ray. The presence of air bubbles (gas) around the intestine or abdominal cavity indicates the signs of NEC. The blood and stool tests, along with the abdominal X-ray, can help the healthcare provider determine the seriousness of the infant's condition.

There are numerous research articles published on diagnostic strategies for identification of NEC. The authors Echevarría Ybargüengoitia J L, et. al in an article “Blood in the feces as an aid to the diagnosis of necrotizing enterocolitis, Boletin Medico del Hospital Infantil de Mexico. 1981 September-October; 38(5):771-776”, reported that it is possible to predict absence of manifestations of NEC when a premature baby does not show gross blood in the stools during the first 21 days of life. When the babies show gross blood in the stools, the frequency of NEC is 63.3% and the higher frequency (89.6%), belongs to the babies with initially microscopic and later gross blood. By the time the blood appears in stool the necrotising enterocolitis is already started and it is too late to prevent the damage caused by NEC.

Some researchers even tried to evaluate clinical biomarkers for identification of NEC. Reference may be made to Yang et al., “Diagnostic value of prealbumin for severe necrotizing enterocolitis. Zhongguo Dang dai er ke za zhi=Chinese Journal of Contemporary Pediatrics. 2016 February; 18(2):105-107”, who investigated the diagnostic value of pre-albumin in neonates with severe NEC and stated that this biomarker could be an important value for the diagnosis of severe NEC (≥IIB) with high sensitivity and specificity. However, Pre-Albumin levels can be high in other inflammatory conditions like septicaemia making it relatively less specific test.

However, currently there is no highly specific and sensitive and prompt method available to detect signs of NEC and to determine if the baby has infection/inflammation/NEC of gut or non-gut related issue. Further, several tests are needed to determine therapeutic strategies to lower signs of NEC.

For the reasons stated above, which will become apparent to those skilled in the art upon reading and understanding the specification, there is a need in the art to provide a method to diagnose and prevent bowel inflammation, infection and necrotizing enterocolitis that facilitates initiation of prompt therapy to limit morbidity and mortality in premature babies. Further, there is need to provide a method that facilitates early management of signs of NEC thereby helping to save lives of premature babies and infants having low birth weight.

The present invention overcomes these lacunae by proposing a unique monitoring and evaluation method that evaluates/tracks gut health, identifies and prevents bowel inflammation, infection and necrotizing enterocolitis of gut or non-gut related issue as detailed hereinafter.

### SUMMARY OF THE INVENTION

Accordingly, the present invention provides a method for monitoring and evaluation of bowel health in premature newborns. The method comprises collecting a stool/fecal sample from the premature newborn and dividing the collected stool/fecal sample into three portions. The microbiomics analysis is performed on a first portion of the stool/fecal sample. Particularly, the microbiomics analysis is performed by subjecting the first portion of the stool/fecal sample to density gradient centrifugation to separate white blood cells, and subjecting the white blood cells to different staining techniques or automated complete blood count analyzer to evaluate percentages of different types of cells, wherein higher percentage of neutrophils as compared to lymphocytes indicates inflammatory conditions/infective processes/risk of necrotizing enterocolitis. The method further comprises diluting a second portion of the stool/fecal sample with a phosphate buffered saline (PBS) to evaluate percentages of cell types. The PBS-diluted portion of the stool/fecal sample is divided into two sub portions. A first sub portion of the PBS-diluted sample is subjected to density gradient centrifugation followed by automated cell counting to evaluate percentages of neutrophils, lymphocytes, and other cell types, and a second sub portion of the PBS-diluted sample is subjected to density gradient centrifugation and flow cytometric analysis to determine percentages of specific cell types. The premature newborns are categorized in low risk and high risk based on percentages of neutrophils, wherein higher percentage of neutrophils indicates frequent testing requirement. The flow-cytometric analysis is performed by incubating the cells of the PBS-diluted sample with at least one cell surface marker, and gating cells to define subpopulations based on expression of the cell surface markers. The cell surface marker is selected from CD45, CD3, CD8, CD11b, TNF-alpha, and CD68. In accordance with the present invention, the flow cytometric analysis provides percentage of CD4 positive (high percentage of helper T cells) indicating immune protection available in the stool/fecal sample. Further, the flow cytometric analysis provides percentage of activated macrophages that indicates inflammatory processes within the stool/fecal sample. The method further comprises subjecting a third portion of the stool/fecal sample to density gradient centrifugation followed by anyone of RT PCR analysis using specific gene signature or RNA extraction and transcriptomic analysis to assess gene expression profiles of gut associated immune cells to evaluate the bowel health of the premature newborn.

### BRIEF DESCRIPTION OF THE DRAWINGS

The detailed description is described with reference to the accompanying figures. In the figures, the left-most digit(s) of a reference number identifies the figure in which the reference number first appears. The same numbers are used throughout the drawings to reference like features and modules.

FIG. 1 is a graph showing taxonomic composition of community at Species level using Stacked Bar plot;

FIG. 2 is a graph showing stool immunophenotype of pre-mature neonates;

FIG. 3 is another graph showing stool immunophenotype of pre-mature neonates;

FIG. 4 shows a heat map of necrotizing enterocolitis (NEC) significant genes;

FIG. 5 shows a heat map of innate immune response;

FIG. 6a-6b show graphs of NEC enrichment; and

FIG. 7 shows NEC volcano plot.

### DETAILED DESCRIPTION OF THE INVENTION

The embodiments herein provide a method for monitoring and evaluation of bowel health in premature newborns. Particularly, the method evaluates/tracks gut health, identifies and prevents bowel inflammation, infection and necrotizing enterocolitis. The method of the present invention involves collecting stool/fecal samples from premature babies and processing the sample using microbiomics, automated cell counting, flowcytometry, and RNA transcriptomics analysis or RT PCR testing to determine risk of necrotizing enterocolitis.

The methods described herein are explained using examples with specific details for better understanding. However, the disclosed embodiments can be worked on by a person skilled in the art without the use of these specific details.

Throughout this application, with respect to all reasonable derivatives of such terms, and unless otherwise specified (and/or unless the particular context clearly dictates otherwise), each usage of:
“a” or “an” is meant to read as “at least one.”
“the” is meant to be read as “the at least one.”
   References in the specification to “one embodiment” or “an embodiment” mean that a particular feature, structure, characteristic, or function described in connection with the embodiment is included in at least one embodiment. The appearances of the phrase “in one embodiment” in various places in the specification are not necessarily all referring to the same embodiment.

Hereinafter, embodiments will be described in detail. For clarity of the description, known constructions and functions will be omitted.

According to a preferred embodiment, the present invention provides a method for monitoring and evaluation of bowel health in premature newborns. Particularly, the method evaluates/tracks gut health, identifies and prevents bowel inflammation, infection and necrotizing enterocolitis.

At first step, the method comprises collecting stool/fecal samples from prematurely born newborns in a regular interval to monitor gastrointestinal (GI) health, inflammation/risk of NEC.

At second step, the method comprises dividing the collected stool/fecal samples in three parts/portions.

At third step, the method comprises performing microbiomics analysis on first part of the stool/fecal sample. The stool microbiomics analysis is performed as frequently as needed. Particularly, the first part of the stool/fecal sample is processed using density gradient centrifugation, particularly Ficoll centrifugation, to separate white blood cells (WBC). These WBC are subjected to different staining techniques or automated complete blood count analyser to evaluate percentages of different types of cells. Higher percentage of neutrophils versus lymphocytes indicates inflammatory conditions/infective processes/risk of necrotizing enterocolitis. In addition, significant changes in percentage of neutrophils from baseline samples to the subsequent samples can be diagnostic of gut inflammatory changes. Accordingly, actions can be made to prevent further damage by either providing gut rest or antibiotics or appropriate probiotic.

In an implementation according to an embodiment of the present invention, the microbiomics analysis comprises 16S ribosomal RNA (rRNA) gene sequencing for microbiome profiling. Particularly, the 16S rRNA gene sequencing is performed by extracting DNA from the stool/fecal sample. Thereafter, 16S DNA libraries are prepared targeting V3-V4 regions of the bacterial 16S rRNA gene using specific primers. The 16S DNA libraries are then amplified via polymerase chain reaction (PCR), and barcodes and sequencing adapters are added via a second round of PCR. The prepared libraries are then evaluated, quantified, normalized, and pooled followed by sequencing the pooled libraries.

In accordance with the present invention, the microbiome analysis includes data processing, normalization, and various analytical methods which are used to assess microbial community composition and diversity in samples. These methods are described hereinafter:

The data processing involves reading abundance count data table, data integrity check and data filtering. In an exemplary embodiment, the abundance count data consists of 16 samples and 657 features/taxa, with 457 operational taxonomic units (OTUs) having counts greater than 2. The data must be in a tab-delimited or CSV format, and a metadata file is required for additional sample information. The data integrity check ensures that the necessary information is collected for analysis, removing constant variables and features present in only one sample. The library size statistics provide insights into the read counts across samples. In this exemplary embodiment, specifications of the library size for inspection are as follows:
Total read counts: 984,253
Average counts per sample: 61,515
Maximum counts per sample: 244,585
Minimum counts per sample: 24,470
   The data filtering aims to remove features with low counts and variance, enhancing the quality of the dataset for analysis. In this exemplary embodiment, after filtering, 360 low abundance features and 10 low variance features are removed, leaving 87 features for further analysis.
   For data normalization, normalization methods are applied to adjust for uneven sequencing depths and improve data comparability. Total sum scaling is performed, while no data rarefaction or transformation is applied. The visual exploration methods are used to visualize the taxonomic composition of community through direct quantitative comparison of abundances. The visual exploration methods allow for the comparison of taxonomic compositions across samples, providing insights into community structure at various taxonomic levels. Particularly, Stacked bar/area plots are used to illustrate the relative abundance of different phyla and genera.
   The community profiling is done using Alpha diversity analysis and Beta diversity analysis. The Alpha diversity measures the diversity within samples or community, considering both the total number of species (richness) and the abundances of the species (evenness). Various indices, including Chaol and Shannon, are used to assess diversity across samples, with statistical significance evaluated through tests. For statistical significance (p-value) considered is 0.11433 (T-test). The Beta diversity compares the diversity or composition between two samples or microbial communities. The distance metrics like Bray-Curtis and UniFrac (Unique Fraction) are utilized. The ordination methods such as Principle Coordinate Analysis (PCoA) visualize the dissimilarity matrix, with statistical significance assessed using Permutational ANOVA (PERMANOVA). In this exemplary embodiment, statistical significance (PERMANOVA) has p-value<0.057. Partial Least Squares Discriminant Analysis (PLSDA) is employed to predict class membership using multivariate regression. A permutation test assesses the statistical significance of class discrimination. Thereafter, heat tree and volcano plot analysis are performed. The heat tree analysis visualizes taxonomic differences using median abundance and statistical tests, while volcano plots combine fold change and p-values to identify significant features. Thereafter, a hierarchical cluster analysis is performed wherein individual samples initially form their own clusters, which are then progressively merged until a single cluster encompasses all samples. This process requires considering two key parameters: the method for measuring similarity or distance between samples (e.g., Bray-Curtis, Shannon, Jaccard, UniFrac) and the chosen clustering algorithm (e.g., average, complete, single, or Ward's linkage). The results are visualized by heatmaps and dendrograms using the tool, with the hclust function in package stat. The univariate analysis is performed to identify differentially abundant features using parametric and non-parametric tests. Features are considered significant based on adjusted p-values, with a cutoff of 0.05. The LDA Effect Size (LEfSe) analysis is used for biomarker discovery, combining statistical significance with biological consistency. In this exemplary embodiment, no significant effects are observed in the analysis. The random forest analysis is performed to identify important features through ensemble classification trees, providing insights into variable importance and classification error rates. The random forest analysis also provides other useful information such as OOB (out-of-bag) error and variable importance measure. This OOB data is then used as test sample to obtain an unbiased estimate of the classification error (OOB error). Variable importance is evaluated by measuring the increase of the OOB error when it is permuted. The outlier measures are based on the proximities during tree construction. In this exemplary embodiment, the OOB error is 0.5 and the features are ranked by the mean decrease in classification accuracy when they are permuted.

At fourth step, the method comprises diluting a second part of the stool/fecal sample with phosphate buffered saline (PBS). The PBS diluted sample is again divided into two sub-parts/sub-portions.

At fifth step, the method comprises subjecting a first part of the PBS diluted sample to density gradient centrifugation, particularly Ficoll centrifugation, followed by an automated cell counter to evaluate percentage of neutrophils vs lymphocytes versus other cell types. The percentage of cell types that will be cut offs to determine presence of inflammation is to be determined.

At sixth step, the method comprises subjecting a second part of the PBS diluted sample to density gradient centrifugation, particularly Ficoll centrifugation, followed by flow cytometric analysis to determine percentage of cell types. The flow cytometric analysis is performed by incubating the cells of the PBS diluted sample with at least one cell surface marker, and gating cells to define subpopulations based on expression of the cell surface markers. The cell surface marker is selected from CD45, CD3, CD8, CD11b, TNF-alpha, and CD68. The percentage of CD4 count versus CD8 count and M1 macrophages versus M2 macrophages that will be cut offs to determine presence of inflammation is to be determined. Particularly, percentage of CD4 positive (high percentage of helper T cells as compared to CD8 cells) provides likely immune protection available in the stool and the flow cytometric analysis provides percentage of activated macrophages (high percentage of M1 versus M2) that indicates likely inflammatory processes within the stool. In an exemplary embodiment of the present invention, it is observed that the activated macrophage percentage in normal samples found around less than 10% vs more than 20% in abnormal samples as compared to resting macrophages. Further, cytotoxic cells T cells (CD8+ve) are lower in normal samples less than 40% while more than 50% in abnormal samples.

At seventh step, the method comprises subjecting a third part/portion of the stool/fecal sample to density gradient centrifugation, particularly Ficoll centrifugation, followed by RT PCR analysis using specific gene signature or RNA extraction and transcriptomic analysis to assess gene expression profiles of gut associated immune cells to evaluate the bowel health of the premature newborn. The signature of RNA analysis to be determined. Particularly, PCR analysis is performed to identify a specific signature of 8 gene MRNA changes from the baseline. The details of these 8 gene MRNA are specified in Table 2.

In an implementation according to an embodiment of the present invention, transcriptomic analysis is performed by obtaining a total RNA preparation from the stool/fecal sample and generating an RNA sequencing (RNA-seq) library from the total RNA preparation using a 3′ mRNA-seq library preparation kit. The RNA-seq library is sequenced to produce sequencing reads and adapter sequences are trimmed from the sequencing reads. The trimmed sequencing reads are aligned to a reference genome and read counts for genes from the aligned reads are obtained. The low expressing genes based on a mean read count threshold are filtered and differential expression analysis is performed on the filtered gene read counts to determine expression profiles. It is understood here that additional flow cytometric markers may be added and additional strengthening of RNA signatures is performed with additional data.

In accordance with the present invention, the method uses stool WBC assays to determine if the baby has infection/inflammation or NEC and percentage of CD4 count and M1 in stool to determine the balance between inflammatory/infectious/NEC versus non-inflammatory conditions.

In an implementation according to the present invention, the stool microbiomics analysis is performed as frequently as needed. Higher percentage of E. coli and lower percentage of Staphylococcus equorum prompts high risk status for NEC. Simultaneous stool cell count is also performed. Higher percentage of neutrophils/M1 cells and lower count of CD4 cells prompts actions to prevent NEC. In addition, Stool mononuclear cell RNA analyses are performed to evaluate if there is any indication of a specific signature created using previous transcriptomics work.

In accordance with the present invention, the premature newborns are categorized in low risk and high risk based on percentages of neutrophils, wherein higher percentage of neutrophils indicates frequent testing requirement. In the context of the present invention, a baseline is considered as results of a first testing which are performed when the premature newborns are admitted in NICU. A tiered approach to monitoring is performed for babies at risk of NEC i.e., baseline testing of all 4 tests. The risk stratification is based upon the baseline results. The high risk newborns/babies are tested more frequently with manual cell counts. If abnormal shift to left is observed (20% higher percentage of neutrophils, band cells as compared to baseline samples), rest of tests may be performed. The newborns/babies with low risk demonstrating symptoms suspicious of NEC also get stratified in high risk category. The therapy is varied based upon changes in signalling. For example, if there is worsening of microbiome but normal cell counts then probiotics are provided. If there is significant change towards inflammation in cell counts are observed the action may include additional testing in the form of traditional blood cultures and blood counts in addition to bowel rest and also antibiotics if peripheral blood count is also abnormal. If a response to viral infection is observed in RT PCR signature the action may include viral panel testing, bowel rest and other supportive management.

The invention is further illustrated hereinafter by means of examples.

### EXAMPLES

### Example 1: DNA Extraction, Library Preparation, and Analysis

### Step 1: DNA Extraction

Fecal/stool samples were subjected to DNA extraction using a commercially available kit (e.g., QIAGEN DNeasy PowerSoil Kit) following the manufacturer's protocol. The extracted DNA was placed into a 10 mM Tris-HCl buffer provided by the kit.

### Step 2: 16S Library Preparation and PCR Amplification

DNA libraries targeting the bacterial 16S V3-V4 regions were prepared using commercially available kit (e.g., Illumina's 16S Metagenomic Sequencing Kit). PCR was performed using the following primers:

Forward
5′-TCGTCGGCAGCGTCAGATGTGTATAAGAGACAGCCTACGGGNGGCW
GCAG,
Reveres
5′-GTCTCGTGGGCTCGGAGATGTGTATAAGAGACAGGACTACHVGGGT
ATCTAATCC

The PCR conditions were set as the following: 95° C. for 3 minutes, 25 cycles at 95° C. for 30 seconds, 55° C. for 30 seconds, 72° C. for 30 seconds, and 72° C. for 5 minutes. The PCR product was then extracted from the reaction mixture with magnetic beads (e.g., Mag-Bind RxnPure Plus, Omega Bio-tek, Norcross, GA). A second round of PCR was then performed to add barcodes and sequencing adapters. The conditions for the second round of PCR were as follows: 95° C. for 3 minutes, 8 cycles of 95° C. for 30 minutes, 55° C. for 30 minutes, 72° C. for 30 minutes, and 72° C. for 5 minutes. The prepared libraries were evaluated using Agilent 2200 TapeStation, quantified with QuantiFluor dsDNA System (Promega), and then normalized, pooled, and sequenced on the MiSeq (Illumina, San Diego, CA).

### Step 3: Metagenomic Profiling

The 16S sequencing data was analyzed by the CosmosID 16s pipeline and database. An operational taxonomic unit (OTU)-based table was generated via QIIME.

### Step 4: Statistical Analysis and Visualization

The visualization and analysis of microbiome data was done using MicrobiomeAnalyst. Particularly, Marker Data Profiling (MDP) tool was utilized and OTU table was uploaded. The microbiome data included a total of 16 samples containing 656 features. The features with a low prevalence and a low variance were filtered out. Thus, 160 features were removed based on low prevalence and 15 low variance features were removed based on IQR and after filtering, 282 features remained. The data was normalized by total sum scaling (TSS) followed by statistical analyses using built-in tools of MicrobiomeAnalyst.

### Example 2: Flow-Cytometric Profiling of Neutrophils, Macrophages, and T-Cell Subpopulations

The cells were suspended in PBS and incubated with following cell surface markers:
CD45: APcCy7, CD3: PerCp, CD8: AF700, CD11b: FITC, TNF alpha: PE, CD68: FITC
   After gating of cells, subpopulations are defined as:

### Neutrophils (CD11b):

CD11b⁺ TNF-α⁺ are classified as “activated neutrophils” (N1).
CD11b⁺ TNF-α⁻ are classified as “resting neutrophils” (N2).

### Macrophages (CD68):

CD68⁺ TNF-α⁺ are classified as “active macrophages.”
CD68⁻ TNF-α⁺ are classified as “not active macrophages.”

### T Cells:

CD8⁺ cells are cytotoxic T cells
CD3⁺ cells helper T cells
   Following table provides details of the flow cytometer set up to detect different fluorescent markers (fluorophores) based on the lasers, detectors, and band pass filters for the analysis in accordance with the present invention:

TABLE 1
ThermoFisher Attune NxT Configuration
Flow Cytometry Core Facility - Georgia Cancer Center -
Augusta University

| | | Band Pass | |
|---|---|---|---|
| Lasers | Detector | Filter | Example Fluorophore(s) |
| Violet | VL1 | 450/40 | DAPI/Pacific Blue/AF405/BD V450/ |
| (405 nm) | | | BV421 |
| | VL2 | 525/50 | BV510/AmCyan |
| | VL3 | 610/20 | BV570/Pacific Orange/BV605/ |
| | | | Qdot 605 |
| | VL4 | 660/20 | BV650/eFluor 650NC/Qdot 655 |
| | VL5 | 710/50 | BV711 |
| | VL6 | 780/60 | BV750/BV785/BV786 |
| Blue | BL1 | 530/30 | FITC/AF488/GFP/CFSE |
| (488 nm) | BL2 | 695/40 | PerCP/PerCP-Cy5.5/PE-Cy5.5 |
| Yellow | YL1 | 585/15 | PE/PI |
| (561 nm) | YL2 | 620/15 | PE-Texas Red/PE-eFluor610/ |
| | | | mCherry |
| | YL3 | 780/60 | PE-Cy7/Qdot 800 |
| Red | RL1 | 670/14 | APC/AF647/Qdot 655/eFluor 660 |
| (640 nm) | RL2 | 720/30 | AF680/AF700 |
| | RL3 | 780/60 | APC-Cy7/APC-H7/APC eFluor 780 |

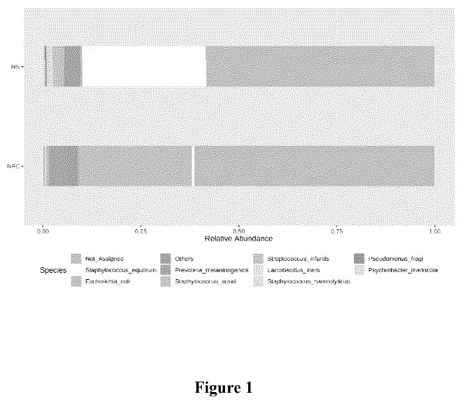

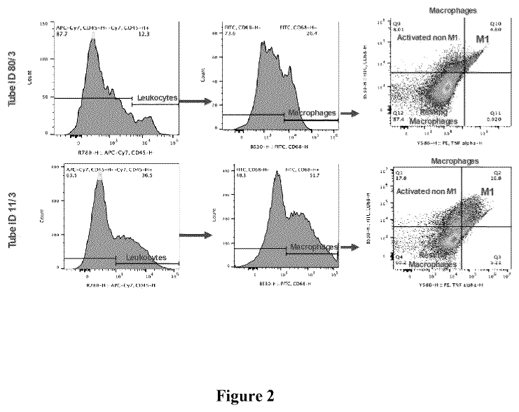

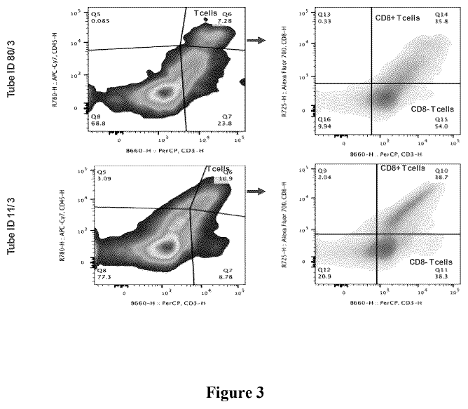

FIG. 1 shows a graph of taxonomic composition of community at Species level using Stacked Bar plot. This graph displays the relative abundance of different bacterial species in two groups: “NN” (“Neonatal Normal” or healthy neonates/controls) and “NEC” (Necrotizing Enterocolitis patients).
FIG. 2 is a graph showing stool immunophenotype of pre-mature neonates. This plots strongly indicate a pro-inflammatory macrophage response in NEC. The increase in M1 macrophages in the NEC sample (Tube ID 11/3) is consistent with the gene expression data showing upregulation of innate immune response genes and inflammatory pathways. This cellular data provides direct evidence of the immune cell phenotype contributing to the inflammation observed in NEC.
FIG. 3 is another graph showing stool immunophenotype of pre-mature neonates. This graph analyzes T-cell populations, specifically identifying CD3⁺ T-cells and then further differentiating them into CD4⁺ and CD8⁺.
Together, all these graphs provide a comprehensive picture of NEC involving gut dysbiosis leading to a robust and potentially dysregulated immune response characterized by M1 macrophage activation and a shift towards cytotoxic CD8⁺ T-cells, all contributing to the pathogenesis of the disease. Most importantly tracking of the gut health would be important and observing drastic changes from the baseline cell percentages would be biomarkers of inflammation.

### Example 3: RNA Extraction and Transcriptomic Analysis

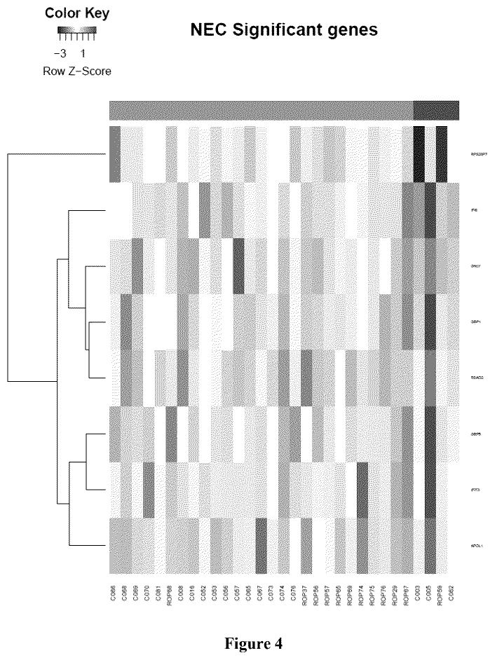

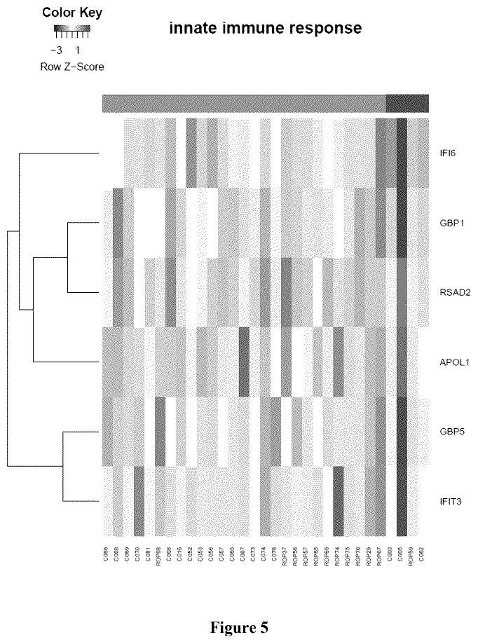

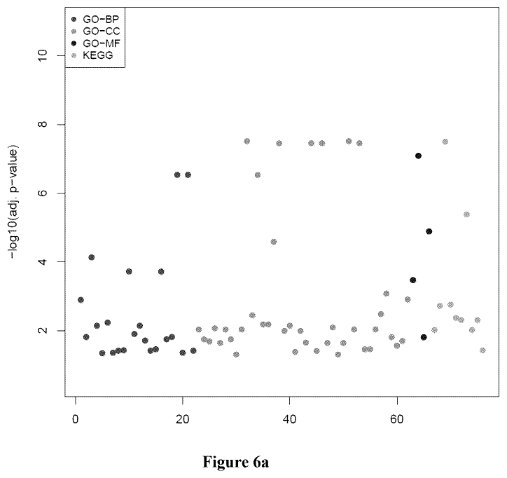

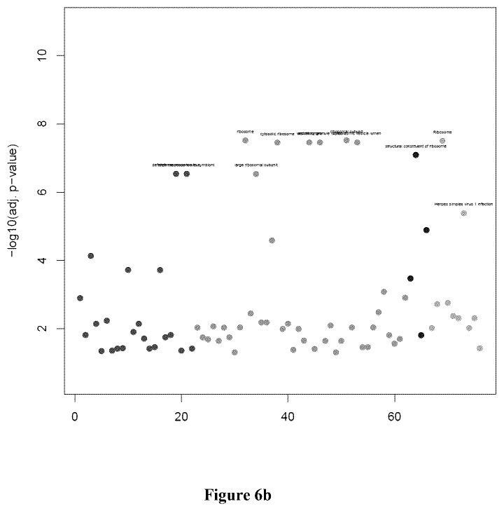

Mononuclear cells were separated using Ficoll gradient centrifugation for total RNA isolation. RNA was isolated using the Trizol method according to the manufacturer's instructions. The quality of the RNA preparations was assessed by measuring absorbance at 260 and 280 nm using a spectrophotometer (e.g., Helios-Gamma, Thermo Spectronic, Rochester, NY). The isolated RNA was submitted to the Genome Sciences Core at Wayne State University for RNA sequencing (RNA-seq). An aliquot of the RNA was assessed by microfluidics using the ScreenTape for the Agilent 2200 TapeStation. The electrophoretogram, RNA Integrity Number (RIN), and the ratio of the 28S:18S RNA bands were collectively examined to determine overall quality of the RNA.
RNA-seq was used to determine expression profiles. Lexogen's QuantSeq 3′ mRNA-seq Library Prep Kit (FWD for Illumina) was utilized for building RNA-seq libraries using 100 ng of total RNA in 5 μl of nuclease-free ultrapure water. Libraries were quantified on the Qubit and Agilent 2200 TapeStation using the DNA High Sensitivity Screen tape. The barcoded libraries were multiplexed at equimolar concentrations and sequenced (75 bp reads; >5M reads per sample) on the NovaSeq 6000. Data was demultiplexed using Illumina's CASAVA 1.8.2 software.
Statistical and bioinformatics analyses: The adapter sequences were trimmed using cutadapt. The sequences were aligned to the human genome (hg38) using STAR, and read counts were obtained. From 31,559 genes identified, low expressing genes (mean read counts less than ten) were removed from further analysis, resulting in 11,399 genes. The differential expression analysis and GSEA (Geneset enrichment analysis) were performed using DESeq2 and Clusterprofiler respectively. For GSEA analysis, GO (gene ontology) and KEGG (Kyoto encyclopedia of genes and genomes) database were used. For visualization, count data were normalized with variance stabilizing transformation.
FIG. 4 shows NEC Significant genes plot which indicates that the heatmap displays the expression patterns of genes that were found to be statistically significant in a differential expression analysis related to NEC. In this graph, different colours are used to visualize gene expression data for example red colour indicates higher gene expression (upregulated), blue colour indicates lower gene expression (downregulated), white/light colours indicate expression levels close to the mean or average. FIG. 5 shows innate immune response indicating that the genes displayed in this heatmap are specifically involved in the innate immune response and were found to be significant in the NEC context.
FIGS. 6a-6b show enrichment plots, specifically designed to visualize the results of a Gene Ontology (GO) and KEGG pathway enrichment analysis. These plots suggest that changes in ribosomal function/protein synthesis, a robust immune/inflammatory response (interferon-gamma signaling), and potentially pathways related to viral infection are significant features of the disease state.
Time for stool microbiome testing: Complicated stool microbiome test results typically take between 2 to 6 weeks to be returned, according to Healthline and Houston Methodist. The exact timeframe can vary depending on the specific testing company and the type of analysis used. Some companies may offer results within 1-2 weeks, while others may take up to 4-6 weeks. The simplified analysis recommended in accordance with the method of the present invention, time is significantly cut down for microbiome testing to less than two weeks as low as two days. Those babies that have abnormal microbiome putting them at high risk of NEC would need more frequent monitoring stool flow cytometry and RNA signature testing.
Time for stool automatic cell count testing: Automated cell counters can provide blood cell counts in a matter of seconds, typically between 9 and 30 seconds. Manual counting, in contrast, can take several minutes per sample. Making it fastest gut health test available. Helping with tracking of gut health rapidly making decision making easy.
Time for stool flow cytometry: This test can be conducted within a few hours adding to specificity of decision making. A shift of abnormal cell counts on automated cell counting procedure can trigger both or either flow cytometry or RNA signature testing based upon severity of shift.
Table 2 below provides details of specific signature of 8 gene MRNA changes from the baseline.

TABLE 2

| | Gene | | Base | log2Fold | | | | |
|---|---|---|---|---|---|---|---|---|
| | Symbol | Function | Mean | Change | lfcSE | Stat | pvalue | padj |
| GBP1 | Guanylate | Immune | 96.64795 | 4.293086 | 0.733568 | 5.852331 | 4.84730228932849e−09 | 4.79155831300121e−05 |
| | Binding | response | | | | | | |
| | Protein 1 | related to | | | | | | |
| | | interferon | | | | | | |
| | | Gamma | | | | | | |
| IFI6 | Interferon | Antiviral | 127.9863 | 3.393557 | 0.725 | 4.680769 | 2.85800759007157e−06 | 0.014126 |
| | alpha- | defense | | | | | | |
| | inducible | and cell | | | | | | |
| | protein 6 | survival | | | | | | |
| GNLY | Granu- | Antimi- | 120.4313 | 2.979144 | 0.654145 | 4.554259 | 5.25704772532049e−06 | 0.015478 |
| | lysin | crobial | | | | | | |
| | | protein | | | | | | |
| | | that is | | | | | | |
| | | released | | | | | | |
| | | from | | | | | | |
| | | cytotoxic | | | | | | |
| | | granules | | | | | | |
| | | upon | | | | | | |
| | | antigen | | | | | | |
| | | stimu- | | | | | | |
| | | lation | | | | | | |
| | | from T | | | | | | |
| | | cells and | | | | | | |
| | | Natural | | | | | | |
| | | Killer | | | | | | |
| | | cells | | | | | | |
| IFIT3 | As | A crucial | 55.34792 | 4.299401 | 0.956098 | 4.496822 | 6.89767681262689e−06 | 0.015478 |
| | interferon- | role in the | | | | | | |
| | induced | innate | | | | | | |
| | protein | immune | | | | | | |
| | with | response | | | | | | |
| | tetratrico- | to viral | | | | | | |
| | peptide | infections. | | | | | | |
| | repeats 3 | | | | | | | |
| GBP5 | Guanylate | An inter- | 93.58407 | 3.861752 | 0.863964 | 4.469804 | 7.82912858276107e−06 | 0.015478 |
| | Binding | feron- | | | | | | |
| | Protein 5, | inducible | | | | | | |
| | | GTPase | | | | | | |
| | | involved | | | | | | |
| | | in innate | | | | | | |
| | | immunity | | | | | | |
| | | and inflam- | | | | | | |
| | | mation. A | | | | | | |
| | | biomarker | | | | | | |
| | | of inflam- | | | | | | |
| | | mation | | | | | | |
| RSAD2 | Viperin | A protein | 48.51342 | 3.422512 | 0.781864 | 4.377374 | 1.20117710542968e−05 | 0.018252 |
| | | with | | | | | | |
| | | antiviral | | | | | | |
| | | and | | | | | | |
| | | immuno- | | | | | | |
| | | modulatory | | | | | | |
| | | functions. | | | | | | |
| | | Induced | | | | | | |
| | | by viral | | | | | | |
| | | infections | | | | | | |
| RPS28P7 | Pseudo- | Unknown | 1742.38 | −2.4524 | 0.562299 | −4.36137 | 1.29248545000594e−05 | 0.018252 |
| | gene of | significance | | | | | | |
| | the RPS28 | | | | | | | |
| | gene | | | | | | | |
| APOL1 | Apolipo- | Triggered | 28.40566 | 2.787504 | 0.657647 | 4.238601 | 2.24916807074359e−05 | 0.027791 |
| | protein | by various | | | | | | |
| | L1 | factors, | | | | | | |
| | | including | | | | | | |
| | | inflam- | | | | | | |
| | | matory | | | | | | |
| | | stimuli | | | | | | |
| | | like | | | | | | |
| | | interferons | | | | | | |
| | | and IL-1β, | | | | | | |
| | | and | | | | | | |
| | | through | | | | | | |
| | | pathways | | | | | | |
| | | involving | | | | | | |
| | | TLR3 | | | | | | |
| | | signaling. | | | | | | |
| | | Addition- | | | | | | |
| | | ally, | | | | | | |
| | | hypoxia | | | | | | |
| | | and drugs | | | | | | |
| | | that | | | | | | |
| | | stabilize | | | | | | |
| | | hypoxia- | | | | | | |
| | | inducible | | | | | | |
| | | transcription | | | | | | |
| | | factors | | | | | | |
| | | (HIFs) can | | | | | | |
| | | also | | | | | | |
| | | upregulate | | | | | | |
| | | APOL1 in | | | | | | |
| | | kidney | | | | | | |
| | | cells | | | | | | |

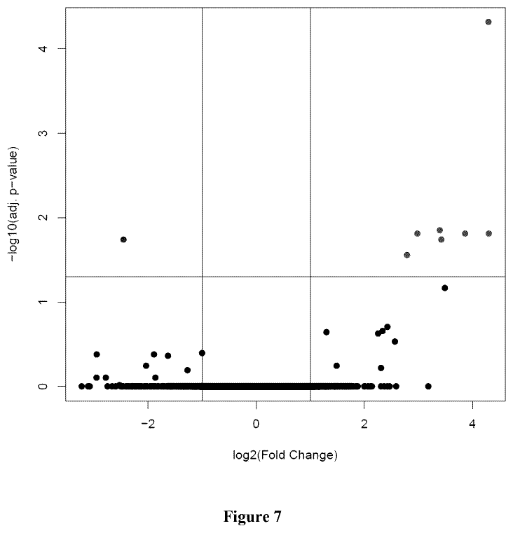

The RNA sequencing study yielded a very small signature of 8 genes as a diagnostic marker of NEC. This test can be conducted within a few hours. Specifically, response to viral infection or inflammatory response regulation is associated with subsequent NEC per current data. Use of specific RNA signature for diagnosis of NEC is a novel finding.
FIG. 7 shows a NEC Volcano plot wherein each point on the plot represents an individual gene or measured entity. This plot helps to quickly identify genes that are significantly upregulated (red dots), significantly downregulated (blue dot), and not significantly differentially expressed (black dots).

Advantage of the present invention according to plurality of embodiments are:
The method allows evaluation and tracking of gut health without needing to draw a blood test and is a non-invasive method.
The method diagnoses and prevents bowel inflammation, infection and necrotizing enterocolitis that facilitates initiation of prompt therapy to limit morbidity and mortality in premature babies.
The method facilitates early management of signs of NEC thereby helping to save lives of premature babies and infants having low birth weight.

In some embodiments, one or more operations have been described as being performed by or otherwise related to certain components, devices or entities, the operations may be performed by or otherwise related to any component, device or entity.

Further, the operations need not be performed in the disclosed order, although in some examples, an order may be preferred. Also, not all functions need to be performed to achieve the desired advantages of the disclosed method, and therefore not all functions are required.

While select examples of the disclosed method have been described, alterations and permutations of these examples will be apparent to those of ordinary skill in the art. Other changes, substitutions, and alterations are also possible without departing from the disclosed method in its broader aspects.

I claim:

1. A method for monitoring and evaluation of bowel health in premature newborns, the method comprising:
collecting a stool/fecal sample from the premature newborn;
dividing the collected stool/fecal sample into three portions;
performing microbiomics analysis on a first portion of the stool/fecal sample;
diluting a second portion of the stool/fecal sample with a phosphate-buffered saline to evaluate percentages of cell types; and
subjecting a third portion of the stool/fecal sample to density gradient centrifugation followed by anyone of RT PCR analysis using specific gene signature or RNA extraction and transcriptomic analysis to assess gene expression profiles of gut associated immune cells to evaluate the bowel health of the premature newborn.

2. The method as claimed in claim 1, wherein the microbiomics analysis is performed by,
subjecting the first portion of the stool/fecal sample to density gradient centrifugation to separate white blood cells; and
subjecting the white blood cells to different staining techniques or automated complete blood count analyser to evaluate percentages of different types of cells, wherein higher percentage of neutrophils as compared to lymphocytes indicates inflammatory conditions/infective processes/risk of necrotizing enterocolitis.

3. The method as claimed in claim 1, wherein the phosphate-buffered saline diluted portion of the stool/fecal sample is divided into two sub-portions, wherein
a first sub-portion of the phosphate-buffered saline diluted sample is subjected to density gradient centrifugation followed by automated cell counting to evaluate percentages of neutrophils, lymphocytes, and other cell types; and
a second sub-portion of the phosphate-buffered saline diluted sample is subjected to density gradient centrifugation and flow cytometric analysis to determine percentages of specific cell types.

4. The method as claimed in claim 3, wherein the flow cytometric analysis is performed by incubating the cells of the phosphate-buffered saline diluted sample with at least one cell surface marker, and gating cells to define subpopulations based on expression of the cell surface markers.

5. The method as claimed in claim 4, wherein the cell surface marker is selected from CD45, CD3, CD8, CD11b, TNF-alpha, and CD68.

6. The method as claimed in claim 4, wherein the flow cytometric analysis provides percentage of CD4 positive (high percentage of helper T cells) indicating immune protection available in the stool/fecal sample.

7. The method as claimed in claim 4, wherein the flow cytometric analysis provides percentage of activated macrophages that indicate inflammatory processes within the stool/fecal sample.

8. The method as claimed in claim 3, wherein the premature newborns are categorized in low risk and high risk based on percentages of neutrophils, wherein higher percentage of neutrophils indicates frequent testing requirement.
